(19) United States
(12) Patent Application Publication
(10) Pub. No.: US 2026/0273171 A1
(43) Pub. Date: Sep. 17, 2026

(54) SYSTEMS AND METHODS FOR MEDICAL DEVICE MODULE DETECTION

(71) Applicant: CareFusion 303, Inc., San Diego, CA (US)

(72) Inventors: Jay DAVE, Carlsbad, CA (US); Zunming ZHANG, San Diego, CA (US)

(21) Appl. No.: 19/076,899

(22) Filed: Mar. 11, 2025

(51) Int. Cl. A61M 5/142; A61M 5/168

(52) U.S. Cl. CPC A61M 5/142; A61M 5/16831; A61M 2005/14208

(57) ABSTRACT

Certain aspects of the disclosure provide systems and techniques for operating medical devices. An example method includes obtaining over a period of time, an inrush current of one or more hardware modules connected to the process control unit; determining a respective type of each of the one or more hardware modules based on the inrush current; and operating the process control unit based on the respective type of each of the one or more hardware modules.

### BACKGROUND

### Field

Aspects of the present disclosure relate to medical device operation.

### Description of Related Art

Medical infusion systems are an important part of modern healthcare infrastructure, enabling precise and controlled delivery of therapeutic substances to patients in both acute and chronic care settings. Contemporary infusion systems typically implement a modular architecture characterized by a central controller unit that coordinates multiple independently operating pump modules, each capable of administering different therapeutic agents according to specifically programmed parameters and protocols.

The central controller in modern infusion systems serves as the primary interface through which healthcare providers program, monitor, and manage multiple simultaneous infusion therapies. This controller typically incorporates a user interface display, processing capabilities, and communication interfaces that enable coordination with connected pump modules. The pump modules, which may include various types such as large volume pumps, syringe pumps, or specialized delivery mechanisms, physically and communicatively connect to the central controller through standardized interfaces. Infusion systems characterized by modular architectures provide healthcare facilities with the flexibility to configure systems according to specific patient needs while maintaining centralized control and monitoring capabilities through a single coordinated interface.

However, while these modular architectures provide many benefits in delivering care to a patient, they can also cause difficulty in ensuring that the wrong modules are not connected to the controller.

### SUMMARY

Certain aspects provide a method for operating a medical device. The method includes obtaining over a period of time, an inrush current of one or more hardware modules connected to the controller; determining a respective type of each of the one or more hardware modules based on the inrush current; and operating the controller based on the respective type of each of the one or more hardware modules.

Other aspects provide processing systems configured to perform the aforementioned methods as well as those described herein; non-transitory, computer-readable media comprising instructions that, when executed by a processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more aspects.

### DESCRIPTION OF THE DRAWINGS

The appended figures depict certain aspects and are therefore not to be considered limiting of the scope of this disclosure.

FIG. 1 depicts an example medical device comprising a controller that is configured to measure inrush currents associated with different hardware modules connected to the controller.

FIG. 2 depicts various examples of inrush currents.

FIGS. 3-5 depict various configurations of medical devices and corresponding inrush current measurements.

FIG. 6 depicts a process flowchart for performing module detection based on inrush currents.

FIG. 7 depicts an example architecture for performing module detection using machine learning.

FIG. 8 depicts an example architecture for performing module detection based on a total energy associated with an inrush current.

FIG. 9 depicts an example architecture for performing module detection based on a waveform associated with an inrush current.

FIG. 10 depicts an example infusion system.

FIG. 11 depicts an example flowchart for module detection.

FIG. 12 depicts an example computing device with which aspects of the present disclosure can be performed.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for performing module detection, such as for security in modular medical devices, such as infusion systems. Though certain aspects are discussed with respect to infusion systems, it should be understood that the techniques discussed herein may also be used with other types of medical devices.

System communication in certain modular medical devices, such as infusion systems, may rely on message-based protocols. Message-based protocols facilitate communication by exchanging data (e.g., discrete data packets) between system components, such as to convey operational parameters, status information, control commands, or the like. For example, when a pump module is physically connected to a central controller of an infusion system, it may initiate a discovery sequence by transmitting identification messages to the central controller. These messages may contain information such as the module's type, capabilities, operational parameters, or the like. The central controller may process these identification messages to establish appropriate communication channels for ongoing operation and monitoring. It may be important that only authorized and correctly functioning modules are used with the central controller to provide high-quality patient care. Message-based protocols allow the central controller to identify information about new modules that are connected into the infusion system and determine if they should be allowed to remain connected within the infusion system.

However, message-based communication architectures may present some technical challenges, such as the potential for message interception, unauthorized message injection, or the like. Some technical challenges also relate to difficulty in accounting for user errors, like the wrong module being attached to the controller. For example, a nurse may accidentally attach a small volume pump (SVP) module, syringe module, or patient controlled analgesia (PCA) module instead of the intended large volume pump (LVP) module.

Notably, solutions to mitigating these challenges may need to account for clinical requirements associated with infusion systems. For example, healthcare providers frequently add new pumps when additional medications need to be administered to patient or remove modules that are no longer needed. Thus, infusion systems may need to provide healthcare providers with the ability to quickly arrange pump modules without interrupting system operation. Corresponding authentication processes of those modules may need to be fast and efficient so as to not delay or interrupt urgent patient care.

One approach to increasing security in message-based protocols is the implementation of encrypted channels. Encryption is the process of converting readable data into a scrambled format that can only be decoded back to its original form by authorized parties who have the correct decryption key. Communicating devices will first need to establish and exchange encryption keys. Then when sending a message, a first device uses the encryption key to encode the original message and send the encoded message to a second device. The receiving device uses the corresponding decryption key to decode the message. Encryption processes may introduce latency in communication processes and require complex setup procedures that may impede rapid module deployment or continuous operation. Additionally, encryption in message-based protocols may be computationally expensive, requiring increased memory for storing keys and handling encrypted data, increased processing power, and additional network bandwidth to account for larger message sizes.

Accordingly, certain aspects of the present disclosure are directed to systems and methods for module detection. Such module detection techniques may further allow for fast and efficient implementation of preventative security measures. In particular, in certain aspects, a controller is provided that is enabled with module detection based on analyzing inrush currents. In certain aspects, when a module is attached to the controller, it is powered up through the controller. During this powering-up process, an inrush current corresponding to the module may be measured by the controller. Inrush current may refer to the lead up to and decay from a spike in the current associated with a module while it is powering-up. In certain aspects, based on analyzing the inrush current, the controller is able to automatically detect information about the module. Some types of information that can be automatically determined include whether the module is known or unknown, authorized or not authorized, a hardware type, and/or age of the module. In some aspects, an inrush current may correspond to more than one hardware module, wherein the inrush current can be analyzed to determine information about each of the corresponding hardware modules.

A known module is a module whose identity can be determined and is recognized by the controller while an unknown module is one whose identity cannot be determined and is not recognized by the controller. An authorized module is a module that has been authorized to be used with the controller, while an unauthorized module is a module that has not been authorized to be used with the controller. A hardware type refers to the hardware configuration of the module that enables a specific functionality. Some example hardware types include a volumetric infusion pump, such as large volume pump (LVP) or small volume pump (SVP), a syringe infusion pump, or a patient-controlled analgesia (PCA) pump. The age of the module may be determined based on the time passed since a manufacturing date.

In particular, modules may exhibit different inrush currents based on their hardware components. Different modules may have varying hardware components, such as motors, capacitors, sensors, or the like, and the types and configurations of these hardware components may induce different inrush currents during the powering-up process. Further, the controller may store information that associates different inrush currents with different sets of information corresponding to different modules. The controller may match the inrush current with a set of information corresponding to a module, to determine information about the module. The controller may also be configured to operate the system based on the type of module. Beneficially, in certain aspects, if the module is determined to be unknown or unauthorized, the controller can perform preventative measures to mitigate any potential issues associated with that type of module.

In some aspects, the controller is configured to compare a measured inrush current with stored inrush currents that correspond to known modules. Based on comparing the inrush current to the stored inrush currents, the controller is able to determine a type of the module. In some aspects, this comparison can be accomplished according to one or more methods described herein, including waveform feature analysis, total energy comparison, waveform comparison, and deep learning.

In certain aspects, waveform feature analysis is performed by analyzing specific features of the inrush current waveform and comparing those features with features that are known to correspond to certain types and characteristics of different modules. For example, a clean waveform does not have significant noise (e.g., irregular fluctuations) in the waveform and may be indicative of an authorized module. In another example, a waveform that has a reduced amplitude as compared to a previously measured waveform may indicate an aging module. In certain aspects, total energy comparison is performed by comparing a total energy associated with the inrush current with stored total energies that are known to correspond to certain types of modules. In certain aspects, similarly, waveform comparison is performed by comparing the waveform of the inrush current with stored waveforms known to correspond to certain types of modules. In some aspects, the controller utilizes deep learning algorithms that are configured to analyze inrush current(s) and determine module type(s) and/or other characteristics. In certain such aspects, a database of stored module profiles and corresponding inrush currents may be used to train a machine learning model to perform module detection.

Accordingly, certain aspects of the present disclosure beneficially provide systems and methods that overcome many of the technical problems associated with module detection.

Certain aspects provide techniques that overcome the technical problem of unwanted latency in the detection process associated with encrypting messages in a message-based communication protocol. For example, module detection based on inrush currents may not involve the exchange of encryption keys, nor may it require the encoding and decoding steps associated with encryption processes. Module detection based on inrush currents, therefore, may occur nearly instantaneously (e.g., within milliseconds of hardware modules being connected to the controller). Furthermore, certain aspects provide techniques to analyze an inrush current quickly and efficiently to detect the type of the hardware module shortly after measuring the inrush current.

In certain aspects, because module detection can be performed quickly, such as without increased latency in the detection process, if an unknown or unauthorized module is detected, preventive security measures can be taken immediately and automatically. In particular, in certain aspects, a medical device is configured to stop providing power to an unknown or unauthorized hardware module, stop communicating with the hardware modules, and/or output a warning about the hardware module. The sooner the controller can detect the type of hardware module, the sooner the controller may act to prevent the medical device from becoming compromised.

Additionally, certain aspects provide techniques that overcome the technical problem associated with encryption methods that use significant computational resources, as inrush current analysis may be less computationally complex.

Certain aspects of module detection based on inrush currents may provide a way to learn additional characteristics about a hardware module, such as a hardware type and/or an age of the module. In some aspects, one or more individual hardware components included in the hardware configuration of the module may be determined based on certain corresponding features identified in the inrush current. In certain aspects, these additional characteristics can be used to further validate predicted security threats associated with the hardware module or can be used to tailor the operation of the medical device.

### Example Medical Device

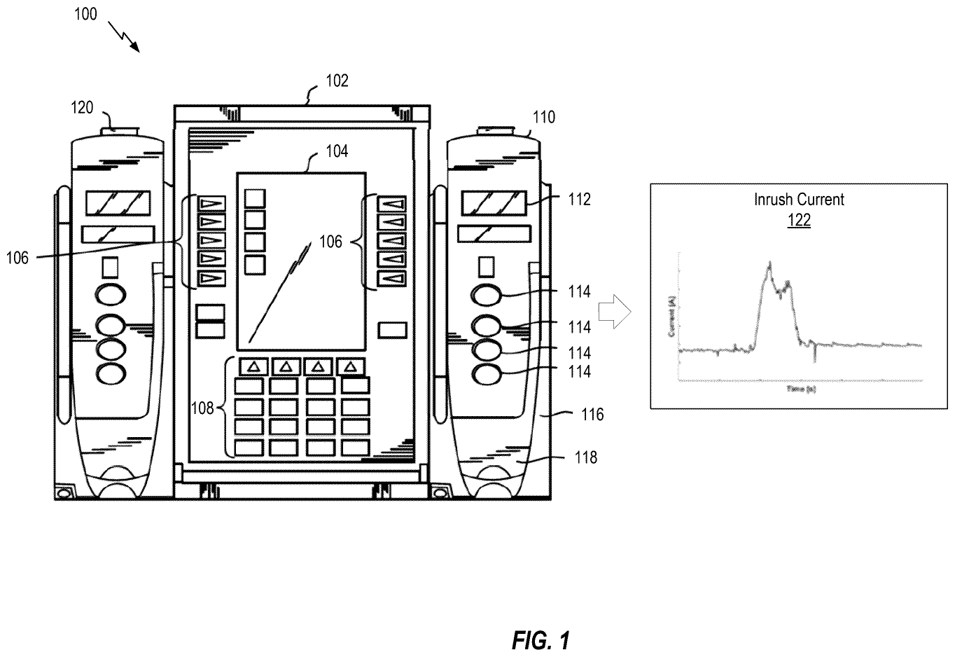

FIG. 1 depicts an example of a medical device 100, which is configured to implement one or more methods described herein for performing module detection. The medical device 100 may include one or more hardware modules, for example, hardware module 110 and hardware module 120, and a controller 102. In some aspects, the controller 102 may be a patient care device (PCD) or patient care unit (PCU). It should be appreciated that while medical device 100 is shown comprising two hardware modules in FIG. 1, medical device 100 may include one hardware module or more than two hardware modules, either connected directly to the controller 102 and/or or connected to one of the hardware modules 110 or 120.

The controller 102 is configured to control operations of the hardware modules 110 and 120, for example, to control infusions to a patient. When integrated with an infusion system, the controller 102 may be further configured to control fluid delivery to the patient and, in some cases, monitor the fluid path for occlusion or air-in-line obstructions. In some aspects, the hardware module 110 and/or the hardware module 120 are configured as pumps. In such a system, each attached pump represents a pump channel of an overall patient care system, including the infusion system.

The controller 102 may further be configured to provide power to the hardware modules 110 and 120. When a hardware module, such as the hardware module 110, is attached to the controller 102, the hardware module 110 is powered up through the controller 102 itself. Because different hardware modules include different hardware components, each hardware module may exhibit a different (e.g., unique) inrush current such as inrush current 122, during the powering-up process.

In certain aspects, inrush current is associated with the maximum instantaneous input current drawn by an electrical device, like hardware module 110, when it is first powered on. The magnitude of an inrush current can be several times higher than the device's normal operating current and can last for a few milliseconds up to several cycles of the input waveform. In certain cases, to measure inrush current accurately, specialized equipment like digital storage oscilloscopes with current probes or dedicated inrush current tests are used. In certain aspects, controller 102 is configured with hardware component(s), such as current probes or dedicated inrush current testers, which are capable of measuring inrush current. Such hardware component(s) may have sufficient bandwidth and high enough sampling rate to capture the brief current spike. In certain aspects, for more comprehensive analysis, controller 102 may be equipped with power quality analyzers that can be used to measure parameters associated with inrush current 122, such as inrush duration, wave shape, the relationship between voltage and current during the powering-up process, or the like.

In certain aspects, the relative uniqueness of an inrush current arises from the distinct hardware components and/or distinct configuration of hardware components within each of the hardware module 110 and 120. Thus, if the hardware modules 110 and 120 include different hardware components, when the hardware module 110 is connected to controller 102, the hardware module 110 will draw a different inrush current than when the hardware module 120 is connected to controller 102. In certain aspects, by measuring the inrush current(s) of hardware module(s) connected to the controller 102, the medical device is able to automatically detect a respective type of each hardware module, such as whether the hardware module is known or unknown, authorized or unauthorized, and/or other characteristics such as age and hardware type of each hardware module because the inrush current can be correlated to the respective type of a hardware module.

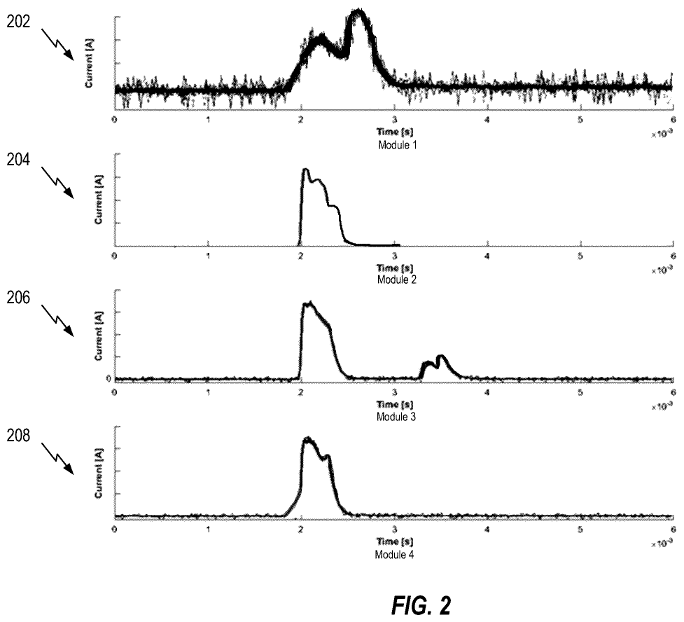

As shown in FIG. 1, the inrush current 122 is represented by a graph showing recorded current values over time. Additional examples of inrush currents are described in further detail with respect to FIG. 2.

The controller 102 may further include a graphical user interface (GUI), such as display 104. In some aspects, display 104 is an information display such as a liquid crystal display, and/or a touchscreen. The display 104 of the controller 102 may be used during set up and/or operating procedures to facilitate control of the medical device 100. For example, display 104 may be configured for entry, editing, and/or display of operational parameters of the hardware modules 110 or 120. The controller 102 may further include one or more softkeys and/or hardkeys to facilitate data entry and commands. For example, the controller 102 has various input devices as shown in FIG. 1, including control keys 106 and a bar code, scanner, or reader for scanning information from an electronic data tag relating to the infusion, the patient, the care giver, or other user.

In some aspects, the display 104 may display the operational parameters of the hardware modules 110 or 120, for example, the infusion rate at which a pump of a hardware module is operating. The display 104 may additionally and/or alternatively, be used to display informational, advisory, alarm, warning, or malfunction messages associated with the hardware modules 110 or 120. The controller 102 may additionally and/or alternatively display one or more of the measurements recorded by a drop sensor, a pressure sensor, and/or a fluid level sensor associated with the hardware module 110 (and/or another hardware module such as hardware module 120).

In some aspects, the controller 102 is configured as an interface between aspects of the medical device 100 and external devices and/or networks, for example, patient monitoring networks, instructional systems (e.g., an electronic medical record system), pharmacy systems, and/or nurse call systems, or as an interface to external equipment such as barcode readers to provide a means of inputting drug and/or patient information from medication or patient records. In some aspects, the controller 102 is configured to provide physical attachment of the medical device 100 to additional structures, such as intravenous pole and/or bedrails.

The controller 102 also has a communications system (not shown) with which it may communicate with external equipment such as a medical facility server or other computer and with a portable processor, such as a handheld portable digital assistant (PDA), or a laptop-type of computer, or other information device that a care giver may have to transfer information as well as to download drug libraries to a controller or hardware module. The communications system may take the form of a radio frequency (RF) system, an optical system such as infrared, a Bluetooth system, or other wired or wireless system. The bar code scanner and communications system may alternatively be included integrally with the hardware module 110, such as in cases where a controller is not used, or in addition to one with the controller. Further, information input devices need not be hard-wired to medical instruments, information may be transferred through a wireless connection as well.

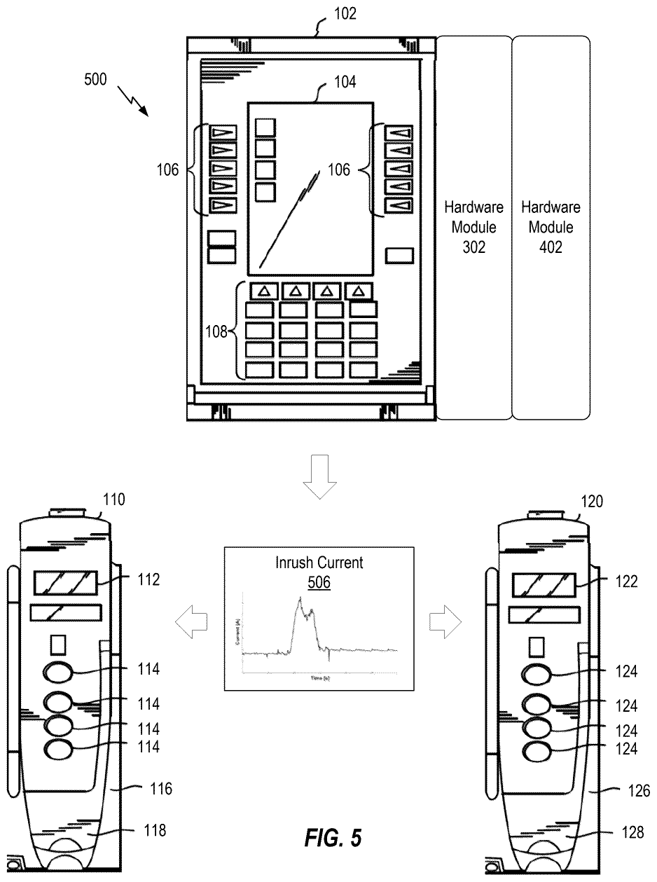

As shown in FIG. 1, the hardware module 110 is attached to the right side of the controller 102. In certain aspects, the controller 102 is used to provide an interface to control the hardware module 110 and the hardware module 120, and in some cases other external devices. For example, the controller 102 may provide most of the operator interface for hardware module 110 and hardware module 120. In some aspects, the hardware module 110 may function as an interface for other devices or modules to connect to controller 102. For example, other devices or modules, including another pump, may be attached to the right side of the hardware module 110, for example, as shown in FIG. 5, and accordingly be connected (e.g., and controlled) to controller 102 via hardware module 110.

The hardware module 110 is shown having a front door 116 and a handle 118 that operates to lock the front door 116 in a closed position for operation and to unlock and open the front door 116 for access to the internal pumping and sensing mechanisms and to load administration sets for the hardware module 110. A display 112, such as an LED display, is located in plain view on the front door 116 and may be used to visually communicate various information relevant to the hardware module 110, such as alert indications (e.g., alarm messages). The display 112 may otherwise be a part of or be coupled to the hardware module 110. Control keys 114 exist for programming and controlling operations of the hardware module as desired. The hardware module 110 also includes audio alarm equipment in the form of a speaker (not shown).

In some aspects, the hardware module 110 and/or the hardware module 120 are configured as pumps. A pump may be an infusion device configured to deliver a substance, (e.g., fluid, nutrients, drug, etc.) to a patient's circulatory system, or epidural space, for example, via an intravenous infusion, subcutaneous infusion, arterial infusion, epidural infusion, etc., or to a patient's digestive system. In such a system, each attached pump represents a pump channel of an overall patient care system. When the front door 116 is open, a tube can be connected with the pump. When the front door 116 is closed, the tube is brought into operating engagement with the pumping mechanism, the upstream and downstream pressure sensors, and the other equipment of the pump.

### Inrush Currents

Based on analyzing an inrush current, such as analyzing one or more features of the inrush current, the controller 102 is able to determine a respective type, such as including one or more characteristics, of one or more hardware modules (e.g., hardware module 110 and/or hardware module 120) connected to the controller 102. Some example features, described in more detail below, that can be analyzed include: waveform shape, total power delivered by the inrush peak, amplitude, a peak current magnitude, the rise time to peak, decay profile shape, settling time, steady-state value, secondary peaks, or the presence of noise or oscillations. Different hardware modules may be associated with certain peak current magnitudes, rise times, decay profile shapes, settling times, steady-state values, the presence and frequency of secondary peaks, amplitude values, or the presence of noise or oscillations.

Thus, in some aspects, the controller 102 is able to identify a respective type of the hardware module (e.g., known or unknown, authorized or unauthorized) and, in some cases, other characteristics such as age and/or hardware type of the hardware module, based on analyzing one or more of the aforementioned features of the inrush current.

Regarding amplitudes of the inrush current, an amplitude of an inrush current of a hardware module may change with the age of the hardware module. For example, an older hardware module (e.g., that was manufactured longer ago) may have a decreased amplitude inrush current as compared to a newer hardware module of the same type. For example, hardware component(s) of a hardware module may age or degrade over time, which may affect the amplitude of the inrush current. Other features of the inrush current, however, may stay the same, such that a type of the hardware module can still be determined based on one or more other features of the inrush current. Thus, in certain aspects, by comparing the amplitude of a known inrush current for a hardware module with an amplitude of a measured inrush current associated with a same type of hardware module (e.g., determined based on one or more other features of the measured inrush current), the approximate age of the hardware module can be determined. In some aspects, the controller is configured to monitor the age of a hardware module and compare the approximated age with an established age threshold, such that when the age of a hardware module increases over a recommended maximum age threshold, the controller is configured to output a warning (or perform some other function) that the hardware module is past the recommended age. The older the hardware module, the more likely that one of the hardware components of the hardware module might fail, causing significant disruption to patient care. Thus, it may be beneficial to monitor the age of a hardware module to prevent issues related to aging hardware component(s).

In certain aspects, peak current magnitude refers to the maximum value that the inrush current reaches and may indicate the maximum stress experienced on the hardware component(s) of the hardware module. The peak current magnitude may also reveal the total capacitance or transformer size. Rise time to peak may refer to the amount of time that the inrush current takes to go from the baseline current to the peak current magnitude. A fast rise to peak may indicate capacitive loading, while a slower rise may indicate transformer behavior. The decay profile shape may refer to the shape of the current as it decreases from the peak current magnitude (or secondary current peak) back down to a baseline current. Exponential decay may correspond to resistor-capacitor (RC) circuits of the hardware module, while oscillatory decay may indicate inductor-capacitor (LC) resonance in the circuit. Additionally, the presence of multiple decay rates may suggest the presence of multiple energy storage units within the hardware module.

In certain aspects, the steady state value is associated with the baseline current before and/or after a current spike in the inrush current. The steady state value to which the inrush current decays may be indicative of the normal operating current of the hardware module. Secondary peaks may occur during the decay process or after the inrush current has decayed back to the steady state value. Secondary peaks in the inrush current may be associated with multiple power supply stages or multiple loads coming online sequentially.

Secondary peaks may also be associated with sniffing devices within or otherwise connected to the hardware module. A sniffing device is a tool used to monitor and analyze signal traffic. Sniffing devices can operate by capturing data packets passing through a network interface or intercepting electronic signals transmitted between devices. While sniffing devices can have legitimate uses, they can also be misused as part of unauthorized network or device monitoring. For example, a user may install a sniffing device between a pumping module and the PCU. Doing so can lead to network security problems, data privacy breaches, or disruption or modification of critical signals (e.g., command and control signals) passing between the devices.

As an additional analysis point, the presence of noise within the current waveform may be indicative of one or more negative factors associated with the hardware module. In some aspects, there may be an expected signal to noise ratio that is unique to a particular type of module. Accordingly, the detected signal to noise ratio associated with a module could be compared against the expected signal to noise ratio to facilitate the detection and characterization of the module, including determining a module type. In some aspects, the noise can be indicative of malfunctioning or degrading hardware components. Alternatively, the noise can be indicative of hardware components that have been refurbished from older models of hardware modules, which can lead to the degradation of the system and may present cybersecurity vulnerabilities. Similarly, the noise may be indicative of a hardware module that has knock-off components that do not function to the standard of authorized hardware modules. Thus, a clean waveform is likely indicative of an authorized, properly operating hardware module.

FIG. 2 depicts various examples of inrush currents. In particular, FIG. 2 depicts an inrush current 202, inrush current 204, inrush current 206, and inrush current 208. Each inrush current is measured over a period of time (e.g., 6 milliseconds) and is different because each inrush current corresponds to a different type of hardware module. For example, inrush current 202, or other example inrush current illustrated, may be associated with an LVP module, a PCA module, a SYR module, or may be associated with an unknown hardware module.

In particular, inrush current 204 may be an example of an inrush current corresponding to a known and authorized hardware module. Inrush current 204 is characterized by a baseline current at 0.0 (A) for about 2 milliseconds, followed by a sharp maximum current spike. A second current spike occurs at approximately 2.2 milliseconds, followed by a plateau at approximately 2.3 milliseconds. The current then decays quickly back to the baseline current at 0 (A). Notably, the current waveform for inrush current 204 is clean and free from noise or significant oscillations. Inrush currents like inrush current 204 may be associated with properly operating hardware modules that are both known and authorized.

Inrush current 202 may be an example of an inrush current corresponding to a known but unauthorized hardware module. Inrush current 202 is characterized by a baseline current hovering near 0.0 (A) for about 2 milliseconds, followed by a semi-sharp maximum current spike just after 2 milliseconds. A second current spike occurs at approximately 2.7 milliseconds. The second current spike is followed by a return to the baseline current hovering near 0.0 (A). Notably, the current waveform for inrush current 204 is very noisy throughout the baseline current measurement and the current spikes.

In some aspects, the noisy current may be indicative of a refurbished hardware module, or a knock-off hardware module obtained from an unauthorized third party. Alternatively, inrush current 202 may be associated with an old age hardware module because the maximum inrush current is under a certain expected maximum inrush current value or range of values. For example, typical inrush currents of new hardware modules may be measured, for example, above 1.0 (A), indicating that one or more of the hardware components of the hardware module are aging if the maximum inrush current that is measured is under 1.0 (A).

Inrush current 206 may be an example of an inrush current corresponding to a known and authorized hardware module with a sniffing device. Inrush current 206 is characterized by a baseline current hovering near 0 (A) for about 2 milliseconds, followed by a sharp maximum current spike. The maximum current spike decays semi-gradually to the baseline current. The baseline current is measured for about 1 millisecond, a second current spike is measured for about 0.5 milliseconds, and a third current spike for about 0.5 milliseconds before the current returns to the baselines current. Notably, the current waveform for inrush current 206 is slightly noisy throughout. The presence of another distinct current spike may be indicative of a sniffing device powering-up after the hardware module is powered up.

Inrush current 208 may be an example of an inrush current corresponding to an unknown hardware module. Inrush current 208 is characterized by a baseline current at 0.0 (A) for about 2 milliseconds, followed by a sharp maximum current spike. A second current spike occurs sharply at approximately 2.2 milliseconds. The current then decays quickly back to the baseline current at 0 (A). However, unlike the similarly shaped current waveform of inrush current 204 which is clean, the current waveform of inrush current 208 is slightly noisy, especially at the baseline current. In this case, the controller has not been able to determine a hardware type of the hardware module. In some aspects, this inability to determine hardware type may factor in the controller determining that the hardware module is unknown and unauthorized.

It should be appreciated that the different features of the inrush currents may be associated with certain types of hardware modules and that the illustrated inrush currents of FIG. 2 and their corresponding analysis are exemplary interpretations and may be interpreted differently based on available data about known hardware modules.

### Example Medical Device Configurations

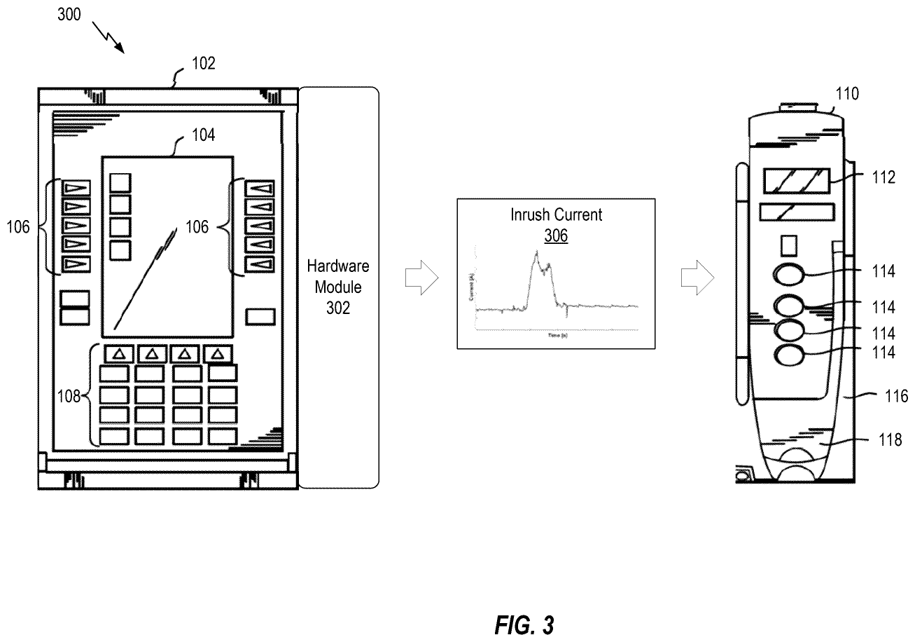

FIGS. 3-5 depict example architectures associated with performing module detection, such as on various configurations of medical devices and corresponding inrush current measurements.

FIG. 3 depicts the controller 102 of FIG. 1 with hardware module 302 that is connected to the controller 102. At the instant when the hardware module 302 is connected to the controller 102, the controller 102 may not know any information about the hardware module 302. When hardware module 302 is connected, the controller 102 measures and records the inrush current 306 associated with a powering-up process of hardware module 302. Here, the inrush current 306 corresponds to a single hardware module connected to the controller 102. Based on the inrush current 306, the controller 102 is able to detect a respective type of hardware module 302. Namely, controller 102 is able to detect that hardware module 302 is hardware module 110.

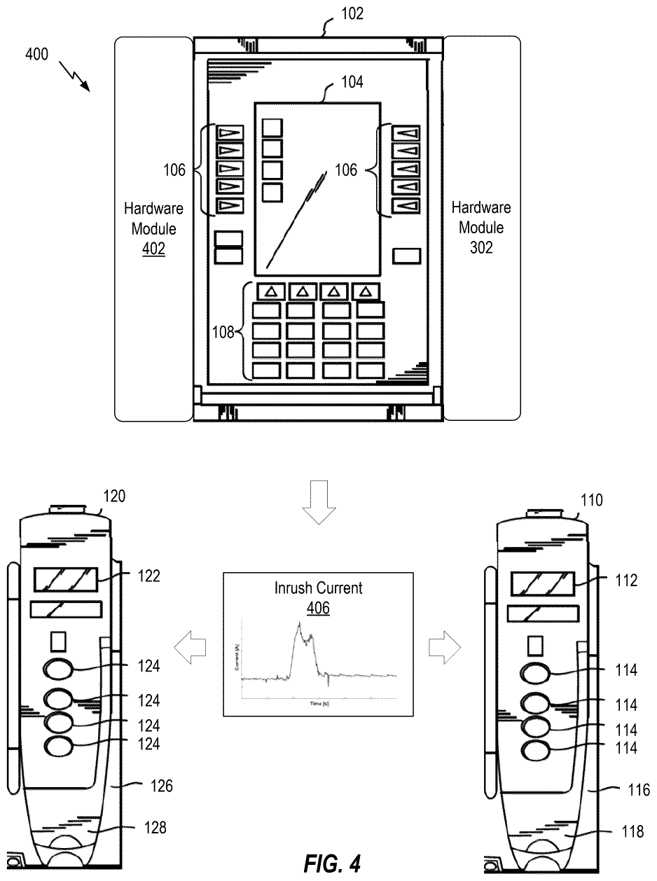

FIG. 4 depicts the controller 102 of FIG. 1 with hardware module 302 of FIG. 3 connected at a right side of the controller 102 and hardware module 402 connected at a left side of the controller 102. In some aspects, inrush current 406 is associated with the powering up-process of both hardware module 302 and hardware module 402. In such aspects, inrush current 406 is a combined inrush current comprising currents generated from both hardware module 302 and hardware module 402. Accordingly, the controller 102 is able to detect the respective type of each hardware module. For example, the controller 102 is able to detect that hardware module 302 is hardware module 110 and hardware module 402 is hardware module 120. In other aspects, inrush current 406 is associated with a powering-up process of hardware module 402 that occurs after the powering-up process of hardware module 302, wherein the controller 102 is configured to detect a respective type of hardware module 302 based on a previous inrush current and then detect a respective type of hardware module 402 based on inrush current 406.

Notably, while the controller 102 is still measuring the current associated with hardware module 302, if hardware module 402 is connected to the controller 102 after hardware module 302, the current associated with hardware module 302 may have returned to a steady-state value, which may be around 0 (A), such that inrush current 406 primarily reflects current values associated with hardware module 402. However, in some aspects the inrush current associated with hardware module 402 is obtained during the same period of time as the inrush current associated with hardware module 302 or during a second period of time that overlaps with or is subsequent to the first period of time associated with hardware module 302.

FIG. 5 depicts the controller 102 of FIG. 1 with hardware module 302 of FIG. 3 connected at a right side of the controller 102 and hardware module 402 of FIG. 4 connected at a right side of the hardware module 302. In some aspects, inrush current 506 is associated with the powering up-process of both hardware module 302 and hardware module 402. In such aspects, inrush current 506 is a combined inrush current comprising currents generated from both hardware module 302 and hardware module 402. Accordingly, the controller 102 is able to detect the respective type of each hardware module. For example, the controller 102 is able to detect that hardware module 302 is hardware module 110 and hardware module 402 is hardware module 120.

In some aspects, the hardware module 302 is configured to measure and record the inrush currents of hardware modules to which it is connected. In such aspects, the controller 102 is configured to measure and record the inrush current of hardware module 302 and hardware module 302 is configured to measure and record the inrush current of hardware module 402. In some aspects, hardware module 302 is also configured to detect a respective type of hardware module 402. In other aspects, hardware module 302 passes the recorded inrush current of hardware module 402 to the controller 102, which then performs the detection of hardware module 402.

### Inrush Current Module Detection

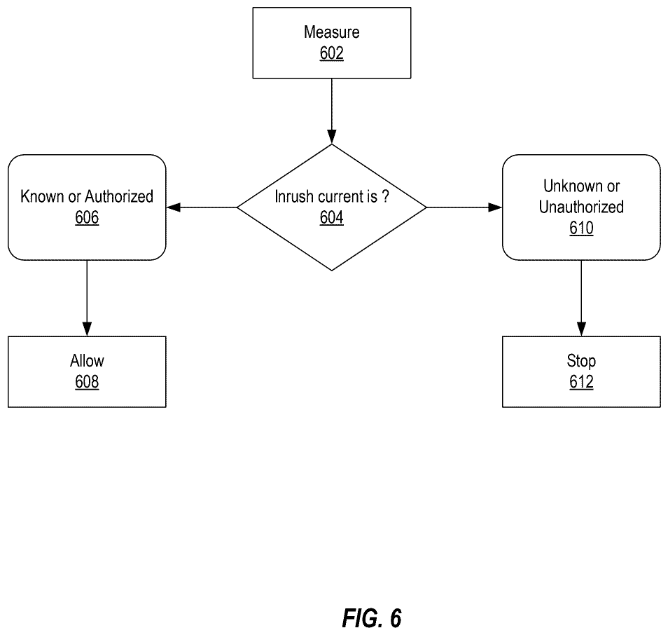

FIG. 6 depicts a process flowchart for performing module detection based on inrush current. In particular, a controller, such as controller 102 of FIG. 1, is configured to measure the inrush current of one or more hardware modules at measure 602 over a period of time. Based on the inrush current, the controller 102 is able to determine a respective type of the one or more hardware modules at decision block 604 (“Inrush current is?”).

In some aspects, the controller 102 determines that a particular hardware module, such as hardware module 302 of FIG. 3, is a known type and/or authorized type (e.g., type 606). When the hardware module is known or authorized, the controller 102 is configured to operate the medical device based on the hardware module being known and/or authorized. For example, the controller 102 may be configured to continue to provide power to the hardware module and/or allow communication between the medical device and the hardware module.

In some aspects, the controller 102 determines that a particular hardware module, such as hardware module 402 of FIG. 4, is an unknown type and/or unauthorized type (e.g., type 610). When the hardware module is unknown or unauthorized, the controller 102 is configured to operate the medical device based on the hardware module being unknown or unauthorized. For example, the controller 102 may be configured to take one or more preventive measures in order to mitigate any cybersecurity risks associated with the unknown or unauthorized hardware module. For example, the controller 102 may be configured to stop providing power to the hardware module and/or stop communication between the medical device and the hardware module.

In some aspects, the controller 102 is configured to output one or more options for operating the at least one of the one or more hardware modules based on the hardware type of the at least one of the one or more hardware modules. These options may be displayed on a user interface associated with the controller 102. Options may be configured as user input fields, output fields related to metrics associated with the operation of the hardware module, selectable icons, network communication settings, or other operation settings.

In certain aspects, the controller 102 may be configured to output a warning that the hardware module is unknown or unauthorized. In some aspects, the controller 102 is configured to output the warning at its user interface, such as display 104 of FIG. 1. Additionally, or alternatively, the controller 102 may be configured to output the warning to the user interface of the unknown or unauthorized hardware module, such as display 112 of FIG. 1. In some aspects, the controller 102 is configured to output a warning to another device connected to the controller 102, another device within the infusion system or overall patient care system, or to a third-party device in communication with the medical device.

In some aspects, the controller 102 is configured to measure a continuous current of one or more hardware modules at measure 602 over a subsequent period of time. In certain aspects, based on the continuous current, the controller 102 is able to determine an updated type of the one or more hardware modules at decision block 604. For example, while a hardware module is connected to the controller 102, the hardware module current draw may exhibit abnormal current behavior which would cause the controller 102 to update the hardware module from an authorized type to an unauthorized type based on the abnormal behavior.

### Machine Learning Module Detection

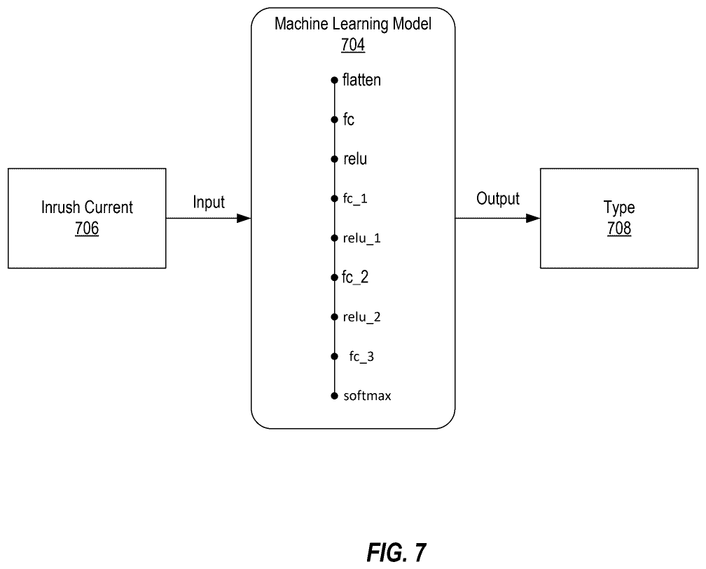

FIG. 7 depicts an example architecture for performing module detection using machine learning. In particular, inrush current 706 (or one or more features derived from inrush current 706) associated with one or more hardware modules, such as hardware module 110 and/or hardware module 120 of FIG. 1, is provided to machine learning model 704. The machine learning model 704 is configured to determine different types of hardware modules based on different inrush currents obtained by a medical device, such as medical device 100 of FIG. 1. In particular, in certain aspects, the machine learning model 704 is configured to generate a model output that indicates the respective type of each of the hardware module(s) based on the inrush current provided to the machine learning model. Further, in certain aspects, the controller is configured to receive the model output and operate the medical device based on the respective type(s) of the hardware module(s) indicated in the model output.

In some aspects, the machine learning model is trained on a database of stored inrush currents that are associated with one or more types of hardware modules. The stored inrush currents are associated with known hardware modules that may be authorized and/or unauthorized. The stored inrush currents may be associated with hardware modules with known ages and known hardware types. By learning the features of the different stored inrush currents, machine learning model 704 may be able to analyze an input inrush current and determine whether the inrush current is associated with a hardware modules that is known or unknown, authorized or unauthorized, and/or identify one or more other features such as age and hardware type.

### Total Energy Module Detection

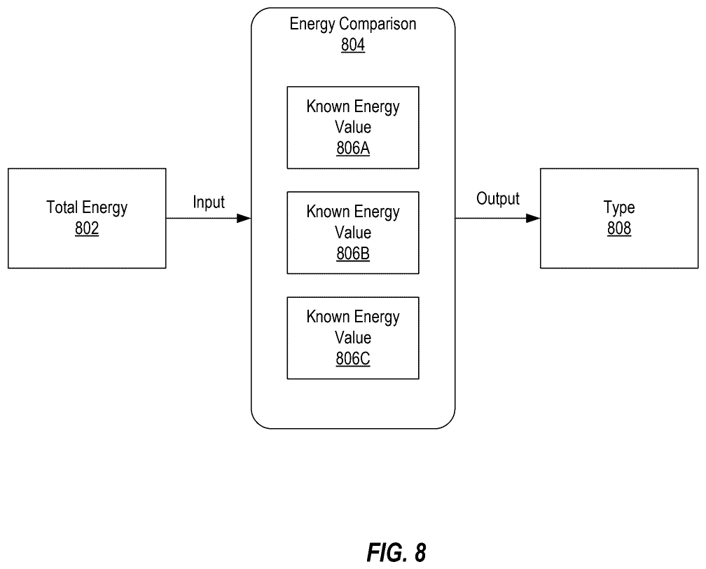

FIG. 8 depicts an example architecture for performing module detection based on a total energy associated with an inrush current. For example, in some aspects, a medical device, such as medical device 100 of FIG. 1, is configured to determine the respective type of one or more hardware modules based on a total energy delivered by the inrush current over a period of time. For example, controller 102 may be configured to measure and record a total energy 802 of a hardware module, such as hardware module 110 of FIG. 1. In some aspects, the controller 102 is configured to perform an energy comparison 804 by comparing total energy 802 with a database of known energy values, such as known energy value 806A, known energy value 806B, and known energy value 806C, associated with different types of hardware modules. Based on comparing total energy 802 with the known energy values, the controller 102 is able to determine a respective type (e.g., type 808) of the one or more hardware modules.

If the total energy 802 matches (e.g., within a threshold, a tolerance, or similarity range) at least one of the known energy values, the controller 102 may be able to determine that the type 808 of the hardware module associated with the total energy 802 is the same type of the hardware module associated with the matching known energy value. In some aspects, the controller 102 determines that the total energy 802 matches one of the known energy values. Accordingly, the type 808 may be known and authorized or known and unauthorized. In some aspects, the controller 102 may be able to determine additional characteristics based on characteristics associated with the matching known energy value, such as age and/or hardware type of the hardware module.

If the total energy 802 does not match at least one of the known energy values, the controller 102 may determine that the type 808 of the hardware module is unknown. In some aspects, when the hardware module is unknown, it is automatically determined to be unauthorized.

In some aspects, total energy 802 is associated with a combination of inrush currents associated with multiple hardware modules. In some such aspects, the database of known energy values may also include known energy values that represent the total energy associated with different combinations of inrush currents associated with different configurations of multiple hardware modules.

### Waveform Module Detection

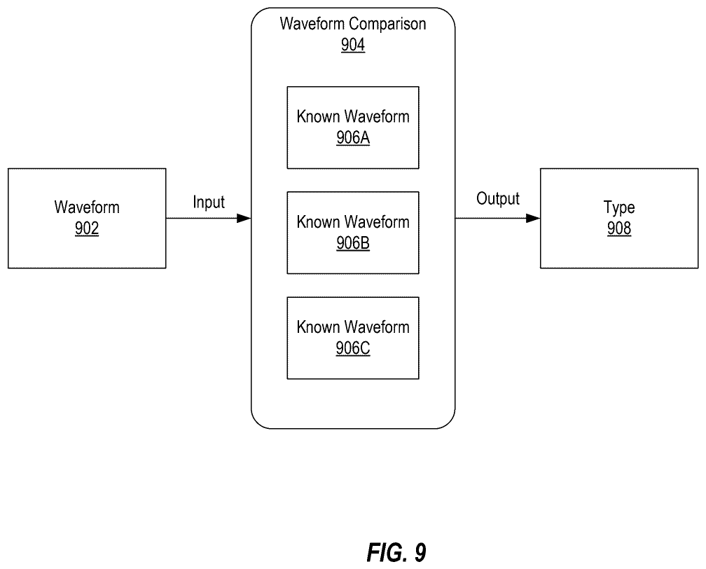

FIG. 9 depicts an example architecture for performing module detection based on a waveform associated with an inrush current. For example, in some aspects, a medical device, such as medical device 100 of FIG. 1, is configured to determine the respective type of one or more hardware modules based on a waveform of the inrush current measured over a period of time. For example, in certain aspects, controller 102 is configured to measure and record a waveform 902 associated with the inrush current of a hardware module, such as hardware module 110 of FIG. 1. In some aspects, the controller 102 is configured to perform a waveform comparison 904 by comparing waveform 902 with a database of known waveforms, such as known waveform 906A, known waveform 906B, and known waveform 906C, associated with different types of hardware modules. Based on comparing waveform 902 with the known waveforms, the controller 102 is able to determine a respective type (e.g., type 908) of the one or more hardware modules.

If the waveform 902 matches (e.g., within a threshold, a tolerance or similarity level) at least one of the known waveforms, the controller 102 is able to determine that the type 908 of the hardware module associated with the waveform 902 is the same type of the hardware module associated with the matching known waveform. In some aspects, the controller 102 determines that the waveform 902 matches one of the known waveforms. In certain aspects the controller 102 may be configured to determine that waveform 902 matches one or more of the known waveforms based on identifying one or more shared features of the matching waveforms, such as a peak current magnitude, rise time to peak, decay profile shape, settling time, steady-state value, secondary peaks, the presence of noise or oscillations, or the like. In some aspects, one or more features of waveform 902 match one or more corresponding features of a known waveform within a tolerance.

Thus, when the waveform 902 matches a known waveform, the type 908 is known and may further determined to be authorized or unauthorized based on whether the type associated with the matching known waveform is authorized or authorized. In certain aspects, the controller 102 may be able to determine additional characteristics based on characteristics associated with the matching known waveform, such as age and/or hardware type of the hardware module associated with the known waveform.

If the waveform 902 does not match at least one of the known waveforms, the controller 102 may be able to determine that the type 908 of the hardware module is unknown. In some aspects, when the hardware module is unknown, it is automatically determined to be unauthorized because the controller 102 does not recognize the hardware module and cannot determine further details about it.

In some aspects, waveform 902 is associated with a combination of inrush currents associated with multiple hardware modules. In some such aspects, the database of known waveforms may also include known waveforms that represent combination waveforms of different inrush currents associated with different configurations of multiple hardware modules.

### Example Infusion System

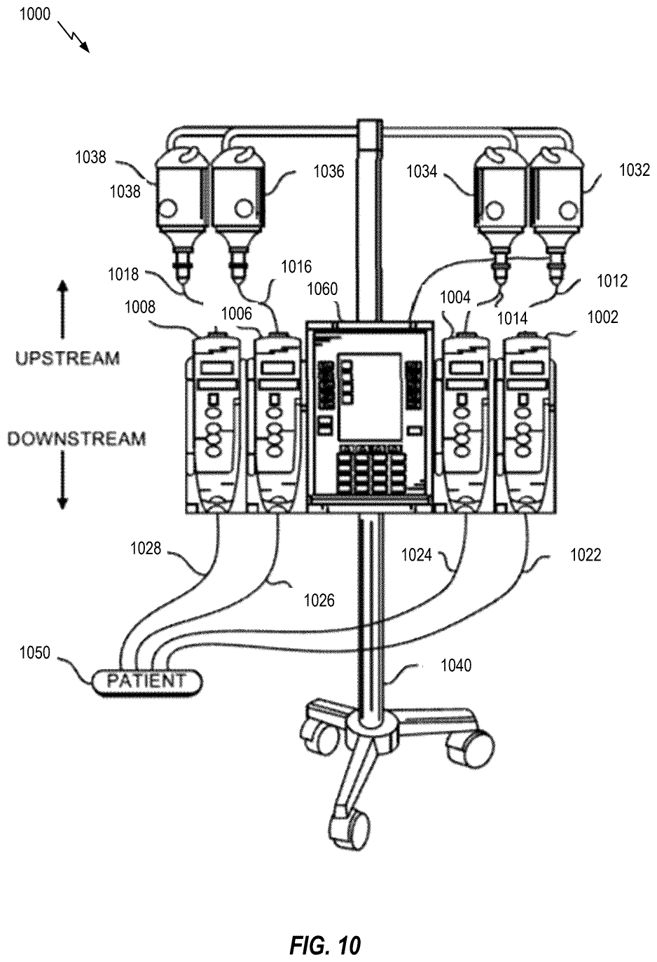

FIG. 10 depicts an example of an infusion system 1000, which may implement methods described herein for module detection. The infusion system 1000 may be coupled to a patient 1050 to administer a substance to the patient 1050 through infusion. The infusion system 1000 may include one or more pumps, for example, pump 1002, pump 1004, pump 1006, and pump 1008. Although a large volume pump is illustrated, other types of pumps may be implemented, such as a peristaltic pump, a small volume pump, a syringe pump, an anesthesia delivery pump, or a patient-controlled analgesic. A pump may be an infusion device configured to deliver a substance, (e.g., fluid, nutrients, drug, etc.) to a patient's circulatory system, or epidural space, for example, via an intravenous infusion, subcutaneous infusion, arterial infusion, epidural infusion, etc., or to a patient's digestive system, for example, via a nasogastric tube (NG), a percutaneous endoscopic gastrostomy tube (PEG), nasojejunal tube (NJ), etc. Each of the pumps 1002, 1004, 1006, or 1008 may include a standard infusion pumping unit, patient-controlled analgesia (PCA) pump, syringe pump, pulse oximeter, invasive or non-invasive blood pressure monitor, electrocardiograph, bar code reader, printer, temperature monitor, RF telemetry link, fluid warmer/IV pump, or high rate IV pump (2000+ ml/hr).

Each of the pumps 1002, 1004, 1006, or 1008 may be fluidly connected with an upstream fluid line 1012, fluid line 1014, fluid line 1016, and fluid line 1018, respectively. Further, each of pump 1002, pump 1004, pump 1006, and pump 1008, may be fluidly connected with a downstream fluid line 1022, fluid line 1024, fluid line 1026, and fluid line 1028, respectively. The fluid lines may be any type of fluid conduit, such as tubing, through which fluid can flow.

Each of fluid supply 1032, fluid supply 1034, fluid supply 1036, and fluid supply 1038, may be a reservoir, for example, as bottles shown, inverted, and suspended above the pumps. Fluid supplies may also take the form of bags, syringes, or other types of containers. The infusion system 1000 may be mounted on a roller stand or intravenous pole 1040.

Infusion system 1000 may further include check valves, drip chambers, valved ports, connectors, and other devices configured to administer a substance.

Each of the pumps 1002, 1004, 1006, or 1008 of infusion system 1000 may be coupled to an infusion pump controller 1060. The infusion pump controller 1060 may be a patient care device (PCD) or patient care unit (PCU). The infusion pump controller 1060 is configured to control operations of the pumps 1002, 1004, 1006, or 1008, for example, to control infusions to patient 1050. The infusion pump controller 1060 may be further configured to the control of fluid delivery to the patient and the monitoring of the fluid path for occlusion or air-in-line obstructions.

The infusion pump controller 1060 may further include a graphical user interface (GUI), for example, an information display such as a liquid crystal display, and/or a touchscreen. The user interface of the infusion pump controller 1060 may be used during set up and/or operating procedures to facilitate control of the infusion system 1000. For example, entry, editing, and/or display of operational parameters of the pumps 1002, 1004, 1006, or 1008. The infusion pump controller 1060 may further include one or more softkeys and/or hardkeys to facilitate data entry and commands. In some aspects, a user interface may display the operational parameters of the pumps 1002, 1004, 1006, or 1008, for example, the infusion rate at which the pump is operating. The user interface may additionally and/or alternatively, be used to display informational, advisory, alarm, or malfunction messages associated with the pumps 1002, 1004, 1006, or 1008.

The infusion pump controller 1060 may further be configured to provide power to the infusion system 1000 and interface between aspects of the infusion system 1000 and external devices and/or networks, for example, patient monitoring networks, instructional systems (e.g., an electronic medical record system), pharmacy systems, and/or nurse call systems, or as an interface to external equipment such as barcode readers to provide a means of inputting drug and/or patient information from medication or patient records. In some aspects, the infusion pump controller 1060 is configured to provide physical attachment of the infusion system 1000 to structures, such as intravenous pole 1040 and/or bedrails, and the like.

### Example Method for Module Detection

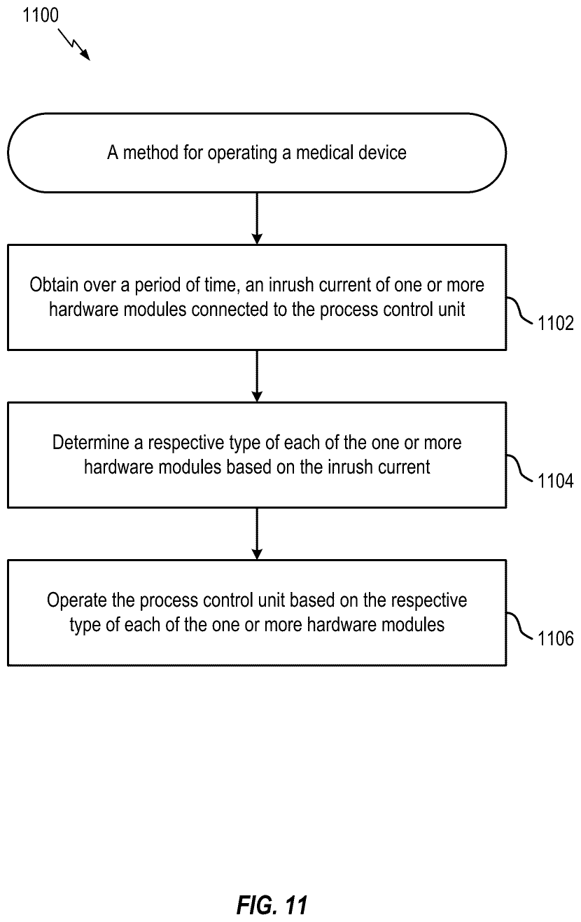

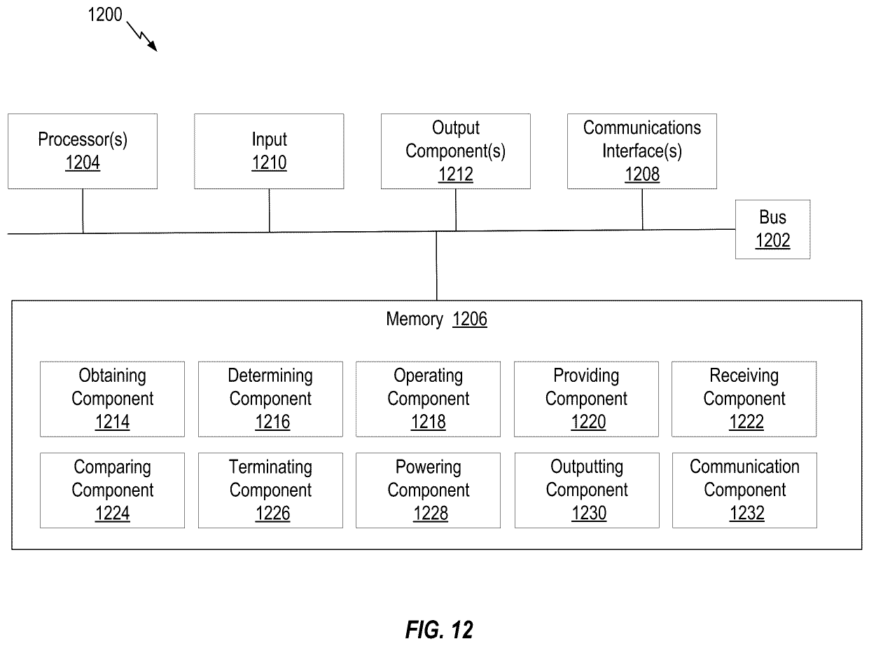

FIG. 11 shows an example of a method 1100 for operating a medical device, such as a controller and/or a computing device 1200 in FIG. 12.

Method 1100 begins at step 1102 with obtaining, over a period of time, an inrush current of one or more hardware modules connected to the controller. In some aspects, step 1102 is performed by obtaining component 1214 of FIG. 12. For example, obtaining component 1214 may obtain, over a period of time, inrush current 306 of FIG. 3 of hardware module 302 connected to controller 102.

Method 1100 then proceeds to step 1104 with determining a respective type of each of the one or more hardware modules based on the inrush current. In some aspects, step 1104 is performed by determining component 1216 of FIG. 12. For example, determining component 1216 may determine the respective type of hardware module 302 of FIG. 3 (e.g., the type of hardware module 110).

Method 1100 then proceeds to step 1106 with operating the controller based on the respective type of each of the one or more hardware modules. In some aspects, step 1106 is performed by operating component 1218 of FIG. 12. For example, operating component 1218 may operate controller 102 of FIG. 3 based on the respective type of hardware module 110.

In some aspects, method 1100 includes providing the inrush current to a machine learning model configured to determine different types of hardware modules based on obtained inrush currents. In such aspects, method 1100 further includes receiving, from the machine learning model, a model output indicating the respective type of each of the one or more hardware modules based on the inrush current provided to the machine learning model. By way of example, providing component 1220 of FIG. 12 may provide inrush current 706 of FIG. 7 to machine learning model 704 and receive model output indicating the type 708 of the hardware module based on inrush current 706.

In some aspects, method 1100 includes determining the respective type of each of the one or more hardware modules based on a total energy delivered by the inrush current over the period of time. By way of example, determining component 1216 of FIG. 6 determines the type 808 of FIG. 8 of the hardware module based on the total energy 802 delivered by the inrush current of the hardware module.

In some aspects, method 1100 includes comparing the inrush current with one or more stored inrush currents that are associated with one or more types of hardware modules, including a first stored inrush current associated with a first type of hardware module. By way of example, comparing component 1224 of FIG. 12 may compare the inrush current (as represented by waveform 902 of FIG. 9) with one or more stored inrush currents, represented by known waveform 906A, known waveform 906B, and known waveform 906C.

In some aspects, the respective type of at least one of the one or more hardware modules is an unknown type or an unauthorized type (e.g., type 610 of FIG. 6) based on the inrush current not matching any of the one or more stored inrush currents.

In some aspects, the respective type of at least one of the one or more hardware modules is the first type based on the inrush current matching the first stored inrush current.

In some aspects, the respective type of a first hardware module of the one or more hardware modules is an unknown type or an unauthorized type (e.g., type 610 of FIG. 6).

In some aspects, method 1100 includes stopping, such as at stop 612 of FIG. 6, communication between the first hardware module and the controller based on the first hardware module being the unknown type or the unauthorized type.

In some aspects, method 1100 includes stopping, such as at stop 612 of FIG. 6, providing power to the first hardware module based on the first hardware module being the unknown type or the unauthorized type.

In some aspects, method 1100 includes outputting a warning that the first hardware module is unknown or unauthorized. For example, outputting component 1230 of FIG. 12 may output a warning to display 104 of FIG. 1.

In some aspects, the respective type of a first hardware module of the one or more hardware modules is a known type or an authorized type (e.g., type 606 of FIG. 6).

In some aspects, method 1100 includes providing, such as at allow 608 of FIG. 6, power to the first hardware module based on the first hardware module being the known type or the authorized type.

In some aspects, method 1100 includes allowing, such as at allow 608 of FIG. 6, communication between the medical device and the first hardware module based on the first hardware module being the known type or the authorized type.

In some aspects, method 1100 includes determining respective one or more characteristics of each of the one or more hardware modules. For example, determining component 1216 of FIG. 12 may determine one or more characteristics of hardware module 110 and 120 of FIG. 1.

In some aspects, determining the respective one or more characteristics of each of the one or more hardware modules includes determining a difference between a first amplitude of the inrush current and a second amplitude of a known inrush current.

In some aspects, the respective one or more characteristics of at least one of the one or more hardware modules include an age.

In some aspects, method 1100 further includes outputting a warning about the age of the at least one of the one or more hardware modules. For example, outputting component 1230 of FIG. 12 may output a warning about the age of hardware module 110 to display 104 of FIG. 1.

In some aspects, the respective one or more characteristics of at least one of the one or more hardware modules include a hardware type.

In some aspects, the hardware type includes a volumetric infusion pump, a syringe infusion pump, or a patient-controlled analgesia (PCA) infusion pump.

In some aspects, method 1100 includes outputting one or more options for operating the at least one of the one or more hardware modules based on the hardware type of the at least one of the one or more hardware modules. For example, outputting component 1230 of FIG. 12 may output options for operating hardware module 110 to display 104 of FIG. 1.

In some aspects, the inrush current is a combined inrush current, such as inrush current 406 of FIG. 4, corresponding to a first hardware module and a second hardware module of the one or more hardware modules, and wherein the method further includes operating the controller based on the respective type of the first hardware module and the respective type of the second hardware module.

In some aspects, the second hardware module is connected directly to the controller, like hardware module 402 of FIG. 4 that is connected to controller 102.

In some aspects, the second hardware module is connected to the first hardware module, like hardware module 402 of FIG. 5 that is connected to hardware module 302.

In some aspects, method 1100 further includes obtaining a second inrush current of a second hardware module connected to a first hardware module of the one or more hardware modules connected to the controller. For example, obtaining component 1214 of FIG. 12 may obtain the inrush current of hardware module 402 of FIG. 5 connected to hardware module 302.

In some aspects, method 1100 further includes determining a second type of the second hardware module based on the second inrush current. For example, determining component 1216 may determine the type of hardware module 402 of FIG. 5 based on the inrush current corresponding to hardware module 402.

In some aspects, method 1100 further includes operating the controller based on the respective type of the first hardware module and the second type of the second hardware module. For example, operating component 1218 of FIG. 12 may operate controller 102 of FIG. 1 based on the type of hardware module 110 and the type of hardware module 120.

In some aspects, the second inrush current is obtained during the period of time or during a second period of time that overlaps with or is subsequent to the period of time. For example, obtaining component 1218 of FIG. 12 may obtain the inrush current associated with hardware module 402 during or after the inrush current for hardware module 302 is obtained.

In some aspects, method 1100 includes obtaining the inrush current during a powering-up process of the medical device.

In some aspects, method 1100 further includes obtaining, over a second period of time, a continuous current of a first hardware module of the one or more hardware modules.

In some aspects, method 1100 further includes determining an updated type of the first hardware module based on the continuous current.

In one aspect, method 1100, or any aspect related to it, may be performed by an apparatus, such as computing device 1200 of FIG. 12, which includes various components operable, configured, or adapted to perform the method 1100. Computing device 1200 is described below in further detail. Note that FIG. 11 is just one example of a method, and other methods including fewer, additional, or alternative steps are possible consistent with this disclosure.

Accordingly, method 1100 overcomes many technical problems associated with module detection.

Method 1100 also overcomes the technical problem of unwanted latency in the detection process associated with encrypting messages in a message-based communication protocol. This is because module detection based on inrush currents does not involve the exchange of encryption keys, nor does it require the encoding and decoding steps associated with encryption processes. Module detection based on inrush currents can occur nearly instantaneously because inrush currents can be measured within milliseconds of hardware modules being connected to the controller. Furthermore, the inrush currents can also be analyzed quickly and efficiently to detect the type of the hardware module shortly after measuring the inrush current.

Because method 1100 can be performed quickly, without increased latency in the detection process, if an unknown or unauthorized module is detected, preventive security measures can be taken immediately and automatically. In particular, the medical device is configured to stop providing power to an unknown or unauthorized hardware module, stop communicating with the hardware modules, and/or output a warning about the hardware module. The sooner the controller can detect the type of hardware module, the sooner the controller can act to prevent the medical device and associated infusion system from becoming compromised.

Additionally, method 1100 the technical problem associated with encryption methods that use significant computational resources. Because the present disclosure avoids message-based communication, the medical device does not require additional memory storage or processing to store and transmit messages, including encoding and decoding encrypted messages.

Method 1100 also achieves additional technical benefits in that module detection based on inrush currents can provide a way to learn additional characteristics about a hardware module, including a hardware type and one or more hardware components, and/or an age of the module. These additional characteristics can be used to further validate predicted security threats associated with the hardware module or can be used to tailor the operation of the medical device.

### Example Computing Device for Smart Module Detection

FIG. 12 depicts an example of a computing device 1200, such as a medical device, which implements various features and processes described herein, such as medical device 100 in FIG. 1. For example, the computing device 1200 may perform one or more steps of any of method 1200. The computing device 1200 may include one or more processors 1204, one or more memories 1206, one or more input components 1210, one or more output components 1212, and one or more communication interfaces 1208. Each of these components may be coupled by a bus 1202.

Computing device 1200 may perform these processes based on one or more processors 1204 executing software instructions stored by a computer-readable medium, such as one or more memories 1206. In certain embodiments, one or more processors 1204 may be programmed/designed/configured to perform these processes. A computer-readable medium (e.g., a non-transitory computer-readable medium) is defined herein as a non-transitory memory device. A memory device includes memory space located inside of a single physical storage device or memory space spread across multiple physical storage devices. Software instructions may be read into one or more memories from another computer-readable medium or from another device via communication interface 1208. When executed, software instructions stored in one or more memories may cause one or more processors 1204 to perform one or more processes described herein.

A memory 1206 may include data storage or one or more data structures (e.g., a database, etc.). Computing device 1200 may be capable of receiving information from, storing information in, communicating information to, or searching information stored in the data storage or one or more data structures in one or more memories 1206.

A memory 1206 may include random access memory (RAM), read only memory (ROM), and/or other types of dynamic or static storage devices (e.g., flash memory, magnetic memory, optical memory, etc.), that stores information and/or instructions for use by one or more processors 1204. For example, a memory 1206 may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

In some aspects, one or more memories 1206 may include obtaining component 1214, determining component 1216, operating component 1218, providing component 1220, receiving component 1222, comparing component 1224, terminating component 1226, powering component 1228, outputting component 1230, and communication component 1232. For instance, in some aspects, obtaining component 1214 may be configured for obtaining over a period of time, an inrush current of one or more hardware modules connected to the process control unit (e.g., as described with reference to step 1102 of FIG. 10. In some aspects, determining component 1216 may be configured for determining a respective type of each of the one or more hardware modules based on the inrush current (e.g., as described with reference to step 1104 of FIG. 10. In some aspects, operating component 1218 may be configured for operating the process control unit based on the respective type of each of the one or more hardware modules (e.g., as described with reference to step 1106 of FIG. 11.

One or more processors 1204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), that may be programmed to perform a function, such as described herein.

One or more input components 1210 may include a component that permits computing device 600 to receive information, such as via user input (e.g., a touch screen display, a keyboard, a keypad, a mouse, a button, a switch, a microphone, etc.). Further, one or more input components may include a sensor for sensing information (e.g., a global positioning system (GPS) component, an accelerometer, a gyroscope, an actuator, etc.).

One or more output components 1212 may include a component that provides output information from computing device 1200 (e.g., a display, a speaker, one or more light-emitting diodes (LEDs), etc.).

Communication interface 1208 may include a transceiver-like component (e.g., a transceiver, a separate receiver and transmitter, etc.) that enables computing device 1200 to communicate with other devices, such as via a wired connection, a wireless connection, or a combination of wired and wireless connections. Communication interface 1208 may permit computing device 1200 to receive information from another device and/or provide information to another device. For example, communication interface 1208 may include an Ethernet interface, an optical interface, a coaxial interface, an infrared interface, a radio frequency (RF) interface, a universal serial bus (USB) interface, a Wi-Fi interface, a cellular network interface, and/or the like.

### Example Clauses

Implementation examples are described in the following numbered clauses:

Clause 1: A method comprising: obtaining over a period of time, an inrush current of one or more hardware modules connected to the process control unit; determining a respective type of each of the one or more hardware modules based on the inrush current; and operating the process control unit based on the respective type of each of the one or more hardware modules.

Clause 2: The method of Clause 1, wherein determining the respective type of each of the one or more hardware modules based on the inrush current comprises: providing the inrush current to a machine learning model configured to determine different types of hardware modules based on obtained inrush currents; and receiving, from the machine learning model, a model output indicating the respective type of each of the one or more hardware modules based on the inrush current provided to the machine learning model.

Clause 3: The method of any one of Clauses 1-2, wherein determining the respective type of each of the one or more hardware modules comprises determining the respective type of each of the one or more hardware modules based on a total energy delivered by the inrush current over the period of time.

Clause 4: The method of any one of Clauses 1-3, wherein determining the respective type of each of the one or more hardware modules based on the inrush current comprises comparing the inrush current with one or more stored inrush currents that are associated with one or more types of hardware modules, including a first stored inrush current associated with a first type of hardware module.

Clause 5: The method of Clause 4, wherein the respective type of at least one of the one or more hardware modules is an unknown type or an unauthorized type based on the inrush current not matching any of the one or more stored inrush currents.

Clause 6: The method of Clause 4, wherein the respective type of at least one of the one or more hardware modules is the first type based on the inrush current matching the first stored inrush current.

Clause 7: The method of any one of Clauses 1-6, wherein the respective type of a first hardware module of the one or more hardware modules is an unknown type or an unauthorized type.

Clause 8: The method of Clause 7, wherein operating the process control unit comprises terminating communication between the first hardware module and the process control unit based on the first hardware module being the unknown type or the unauthorized type.

Clause 9: The method of Clause 7, wherein operating the process control unit comprises stopping providing power to the first hardware module based on the first hardware module being the unknown type or the unauthorized type.

Clause 10: The method of Clause 7, wherein operating the process control unit comprises outputting a warning that the first hardware module is unknown or unauthorized.

Clause 11: The method of any one of Clauses 1-10, wherein the respective type of a first hardware module of the one or more hardware modules is a known type or an authorized type.

Clause 12: The method of Clause 11, wherein operating the process control unit comprises providing power to the first hardware module based on the first hardware module being the known type or the authorized type.

Clause 13: The method of Clause 11, wherein operating the process control unit comprises allowing communication between the medical device and the first hardware module based on the first hardware module being the known type or the authorized type.

Clause 14: The method of any one of Clauses 1-13, wherein determining the respective type of each of the one or more hardware modules comprises determining respective one or more characteristics of each of the one or more hardware modules.

Clause 15: The method of Clause 14, wherein determining the respective one or more characteristics of each of the one or more hardware modules comprises determining a difference between a first amplitude of the inrush current and a second amplitude of a known inrush current.

Clause 16: The method of Clause 14, wherein the respective one or more characteristics of at least one of the one or more hardware modules comprise an age.

Clause 17: The method of Clause 16, further comprising outputting a warning about the age of the at least one of the one or more hardware modules.

Clause 18: The method of Clause 14, wherein the respective one or more characteristics of at least one of the one or more hardware modules comprise a hardware type.

Clause 19: The method of Clause 18, wherein the hardware type comprises a volumetric infusion pump, a syringe infusion pump, or a patient-controlled analgesia (PCA) infusion pump.

Clause 20: The method of Clause 18, wherein operating the process control unit comprises outputting one or more options for operating the at least one of the one or more hardware modules based on the hardware type of the at least one of the one or more hardware modules.

Clause 21: The method of any one of Clauses 1-20, wherein the inrush current is a combined inrush current corresponding to a first hardware module and a second hardware module of the one or more hardware modules, and wherein the method further includes operating the process control unit based on the respective type of the first hardware module and the respective type of the second hardware module.

Clause 22: The method of Clause 21, wherein the second hardware module is connected directly to the process control unit.

Clause 23: The method of Clause 21, wherein the second hardware module is connected to the first hardware module.

Clause 24: The method of any one of Clauses 1-23, further comprising: obtaining a second inrush current of a second hardware module connected to a first hardware module of the one or more hardware modules connected to the process control unit; determining a second type of the second hardware module based on the second inrush current; and operating the process control unit based on the respective type of the first hardware module and the second type of the second hardware module.

Clause 25: The method of Clause 24, wherein the second inrush current is obtained during the period of time or during a second period of time that overlaps with or is subsequent to the period of time.

Clause 26: The method of any one of Clauses 1-25, wherein obtaining, over a period of time, the inrush current of the one or more hardware modules comprises obtaining the inrush current during a powering-up process of the medical device.

Clause 27: The method of Clause 26, further comprising: obtaining, over a second period of time, a continuous current of a first hardware module of the one or more hardware modules; and determining an updated type of the first hardware module based on the continuous current.

Clause 28: An infusion system, comprising: an infusion pump configured to deliver fluid to a patient; and an infusion pump controller configured to perform a method in accordance with any one of Clauses 1-27.

Clause 29: A processing system, comprising: memory comprising computer-executable instructions; and one or more processors configured to execute the computer-executable instructions and cause the processing system to perform a method in accordance with any one of Clauses 1-27.

Clause 30: A processing system, comprising means for performing a method in accordance with any one of Clauses 1-27.

Clause 31: A non-transitory computer-readable medium storing program code for causing a processing system to perform the steps of any one of Clauses 1-27.

Clause 32: A computer program product embodied on a computer-readable storage medium comprising code for performing a method in accordance with any one of Clauses 1-27.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented, or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database, or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A medical device comprising:
a controller comprising one or more memories and one or more processors, wherein the controller is configured to:
obtain over a period of time, an inrush current of one or more hardware modules connected to the controller;
determine a respective type of each of the one or more hardware modules based on the inrush current; and
operate the medical device based on the respective type of each of the one or more hardware modules.

2. The medical device of claim 1, wherein to determine the respective type of each of the one or more hardware modules based on the inrush current, the controller is further configured to:
provide the inrush current to a machine learning model configured to determine different types of hardware modules based on different inrush currents; and
receive, from the machine learning model, a model output indicating the respective type of each of the one or more hardware modules based on the inrush current provided to the machine learning model.

3. The medical device of claim 1, wherein to determine the respective type of each of the one or more hardware modules, the controller is further configured to determine the respective type of each of the one or more hardware modules based on a total energy delivered by the inrush current over the period of time.

4. The medical device of claim 1, wherein to determine the respective type of each of the one or more hardware modules based on the inrush current, the controller is further configured to compare the inrush current with one or more stored inrush currents that are associated with one or more types of hardware modules, including a first stored inrush current associated with a first type of hardware module.

5. The medical device of claim 4, wherein the respective type of at least one of the one or more hardware modules is an unknown type or an unauthorized type based on the inrush current not matching any of the one or more stored inrush currents.

6. The medical device of claim 4, wherein the respective type of at least one of the one or more hardware modules is the first type based on the inrush current matching the first stored inrush current.

7. The medical device of claim 1, wherein the respective type of a first hardware module of the one or more hardware modules is an unknown type or an unauthorized type.

8. The medical device of claim 7, wherein to operate the medical device, the controller is further configured to at least one of:
stop communication between the first hardware module and the controller based on the first hardware module being the unknown type or the unauthorized type;
stop providing power to the first hardware module based on the first hardware module being the unknown type or the unauthorized type; or
output a warning that the first hardware module is unknown or unauthorized.

9. The medical device of claim 1, wherein the respective type of a first hardware module of the one or more hardware modules is a known type or an authorized type.

10. The medical device of claim 9, wherein to operate the medical device, the controller is further configured to at least one of:
provide power to the first hardware module based on the first hardware module being the known type or the authorized type; or
allow communication between the medical device and the first hardware module based on the first hardware module being the known type or the authorized type.

11. The medical device of claim 1, wherein to determine the respective type of each of the one or more hardware modules, the controller is configured to determine respective one or more characteristics of each of the one or more hardware modules.

12. The medical device of claim 11, wherein to determine the respective one or more characteristics of each of the one or more hardware modules, the controller is configured to determine a difference between a first amplitude of the inrush current and a second amplitude of a known inrush current.

13. The medical device of claim 11, wherein the respective one or more characteristics of at least one of the one or more hardware modules comprise an age.

14. The medical device of claim 13, wherein the controller is further configured to output a warning about the age of the at least one of the one or more hardware modules.

15. The medical device of claim 11, wherein the respective one or more characteristics of at least one of the one or more hardware modules comprise a hardware type.

16. The medical device of claim 15, wherein the hardware type comprises a volumetric infusion pump, a syringe infusion pump, or a patient-controlled analgesia (PCA) infusion pump.

17. The medical device of claim 15, wherein to operate the medical device, the controller is further configured to output one or more options for operating the at least one of the one or more hardware modules based on the hardware type of the at least one of the one or more hardware modules.

18. The medical device of claim 1,
wherein the inrush current is a combined inrush current corresponding to a first hardware module and a second hardware module of the one or more hardware modules, and
wherein the controller is further configured to operate the medical device based on the respective type of the first hardware module and the respective type of the second hardware module.

19. The medical device of claim 1, wherein the controller is further configured to:
obtain a second inrush current of a second hardware module connected to a first hardware module of the one or more hardware modules connected to the controller;
determine a second type of the second hardware module based on the second inrush current; and
operate the medical device based on the respective type of the first hardware module and the second type of the second hardware module.

20. A method comprising:
obtaining over a period of time, an inrush current of one or more hardware modules connected to a controller;
determining a respective type of each of the one or more hardware modules based on the inrush current; and
operating the controller based on the respective type of each of the one or more hardware modules.
